(19) United States
(12) Patent Application Publication
(10) Pub. No.: US 2007/0184040 A1
(43) Pub. Date: Aug. 9, 2007

(54) Compositions for delivery of coenzyme Q10

(76) Inventor: Dallas L. Clouatre, Santa Monica, CA (US)

Correspondence Address: Dallas L. Clouatre, 1223 Wilshire Blvd. #761, Santa Monica, CA, 90403-5400, US

(21) Appl. No.: 11/348,062

(22) Filed: Feb. 6, 2006

(51) Int. Cl. A61K 38/43; A61K 31/075; A61K 31/12

(52) U.S. Cl. 424/94.1; 514/690; 514/720

(57) ABSTRACT

The present invention pertains to the beneficial fusibility of ubiquinone (CoQ₁₀) into digestible carrier fats that are solid at room temperature. By melting the CoQ₁₀ into a properly chosen digestible carrier fat(s), recrystallization and phase separation issues are solved and a substrate is supplied for improved action upon CoQ₁₀ by bile acids and lipases. The art taught here allows for the production of CoQ₁₀ products with superior bioavailability, better stability and shelf life, and utilization via tablets, capsules and other dry delivery modalities, either as a single ingredient or as part of complex formulations. This art also can be used to improve CoQ₁₀ inclusion in a variety of soft gelatin capsule formulations.

### BACKGROUND OF THE INVENTION

1. Field of the Invention

The present invention relates to the delivery of coenzyme Q₁₀ and analogs thereof providing increased bioavailability and stability. The invention further makes use of digestible and bioavailable long-chain hydrocarbons as fusible carrier materials.

2. Description of Prior Art

Coenzyme Q₁₀ (ubiquinone) in its chemical structural is related to vitamins E and K. There are CoQs with other numbers, such as CoQ₉, but only CoQ₁₀ appears to be active in humans. Other names for the compound include neuquinon, neuquinone, ubidecarenone and ubiquinone 50. (Molecular formula: C₅₉H₉₀O₄;CAS No: 303-98-0; EINECS No: 206-147-9) Structurally, it is a 2,3-dimethoxy-5-methyl-1,4-benzoquinone with a multi-prenyl side chain, the number of isoprene units depending upon the source from which it is derived, e.g., animal or plant. CoQ₁₀ analogs include reduced and semi-reduced CoQ₁₀ and ubiquinone derivatives. CoQ₁₀ is necessary for energy production, immune response and protection against damage by free radicals.

CoQ₁₀ is part of the mitochondrial electron transport system and is synthesized in all cells. It is essential to the body's production of adenosine triphosphate (ATP). This holds special importance for the heart, which is spectacularly endowed with mitochondria and generally has the body's highest concentration of CoQ₁₀, although this nutrient may be relatively even more abundant in the brain.

Aging reduces access to CoQ₁₀. Although CoQ₁₀ is obtained from the diet (mainly from fatty fish, organ meats, and whole grains) as well as being synthesized in small amounts, both of these routes decline with advancing years. The body's declining capacity to extract and assimilate CoQ₁₀ in later life undoubtedly plays a role in the development of various cardiovascular conditions and cancers.

CoQ₁₀ is present in cell membranes. These membranes are largely constructed of lipids, and CoQ₁₀ itself is lipophilic. Fats are often the targets of free radicals, e.g., the membranes of the blood components known as low density lipoprotein (LDL) cholesterol. At least one important lipid-soluble antioxidant, the alpha-tocopherol form of vitamin E, in excessive amounts can act as a pro-oxidant under certain conditions. Co-supplementation with CoQ₁₀ not only prevents this, but also improves vitamin E's overall protective effects. In order to fully realize the full antioxidant benefits of vitamin E, co-supplementation with CoQ₁₀ or another similar antioxidant may be required.

Not all of CoQ₁₀'s benefits result from its antioxidant and electron transport qualities. It has been recommended in cases of congestive heart failure, cardiac arrhythmias and ischemic injury. These uses are thoroughly established and they have become even more important with the widespread prescription of cholesterol-lowering drugs, especially the statins. Most such drugs act by inhibiting the enzyme HMG-CoA reductase. Inhibition of this enzyme, however, also blocks the biosynthesis of CoQ₁₀ and leads to reduced levels. This fact and the popularity of HMG-CoA inhibitors have been used to explain the increasing prevalence of congestive heart failure in the United States. Statins also increase muscle myopathies and mitochondrial dysfunctions, other indications of CoQ₁₀ depletion. Supplementation with CoQ₁₀, therefore, would be prudent for those taking HMG-CoA inhibitors.

CoQ₁₀ provides many benefits that are hard to explain. For instance, it boosts the capacities of existing immune cells. How this is accomplished is unclear, but at least in part it is the result of reducing the burden on the immune system as a whole. Similarly, although CoQ₁₀ is not a weight-loss agent, it may be important for those overweight or diabetic. Blood tests for CoQ₁₀ indicate that almost 50% of obese subjects are deficient. There is some evidence that this quasi-vitamin can improve pancreatic beta-cell response and glycemic control in proto-diabetic and diabetic individuals.

CoQ₁₀ may help in hypertension. Again, the blood pressure regulating benefits of CoQ₁₀ may be unrelated to its antioxidant benefits. Note that aging causes structural and functional changes to the vascular wall of the cardiovascular system that result in endothelial dysfunction. This endothelial dysfunction is characterized by a decrease in the capacity of the endothelium to properly dilate and may be a significant causative factor in the increased cardiovascular events seen in aging subjects. CoQ₁₀ in clinical work has been demonstrated to enhance endothelial function in patients with ischemic heart disease and heart failure.

CoQ₁₀ is very poorly soluble in most hydrophilic solvents, such as water, and is taken up from the digestive tract as a fatty component. In dry powder form, uptake is notoriously poor, ranging as low as three percent. To overcome this difficulty, various methods have been adopted, such as dissolving the nutrient in a monoterpene or similar substance (U.S. patent applications Ser. Nos. 10/792,648; 10/674,268 and others). Typical monoterpenes include, for example, perillyl alcohol, perillic acid, cis-dihydroperillic acid, trans-dihydroperillic acid, methyl esters of perillic acid, methyl esters of dihydroperillic acid, limonene-2-diol, uroterpenol, and combinations thereof. Other suggested solvents include cetyl meristoleate, dl-alpha tocopheryol acetate, and dimethyl sulfoxide. Limonene, although given GRAS status in the US, nevertheless is a skin irritant and sensitizer. The WHO maintains that its ADI presently is “not specified.”

Yet other techniques proposed and adopted include the use of simple oils, lecithin and other phospholipids, and extreme micronizing. These liquid delivery methods used for CoQ₁₀ can solubilize only up to about 5 percent by weight of the CoQ₁₀ in the “solvent” which then additionally may be held in an aqueous suspension. All of these latter approaches fail to solve the problem that CoQ₁₀ rapidly recrystallizes and the fact that CoQ₁₀ is a solid at body temperature.

Even the casual observer might note that these approaches suffer from the drawback in oral formulations of requiring the use of soft gelatin capsules. Soft gelatin capsules are expensive, have short shelf life, allow for only limited delivery of active ingredients, because of their high moisture content in the gelatin allow for the relatively rapid transmission of oxygen across this barrier, and suffer from numerous other drawbacks. Moreover, placing a number of ingredients in solution or even suspension in water or oil allows for interactions not found in dry blends. Therefore, it is clear that there exists a need in the art for an improved methodology for the delivery of increased amounts of bioavailable CoQ₁₀.

### SUMMARY OF THE INVENTION

The present invention pertains to the surprising discovery that ubiquinone (CoQ10) can be melted beneficially into digestible carrier fats that are solid at room temperature. Despite intense interest in the delivery of CoQ10 in the pharmaceutical and nutritional industries, this approach has not been applied prior to the current invention. Until the present discovery, techniques proposed and adopted included the use of simple oils, lecithin and other phospholipids in oil or oil/water emulsions, and extreme micronization. These liquid delivery methods used for CoQ10 can solubilize only up to about 5-10 percent by weight of the CoQ10 in the “solvent” which then additionally may be held in an aqueous suspension. Another approach is to dissolve the ubiquinone in a monoterpene. These approaches are cumbersome and expensive, with numerous drawbacks as indicated above. By melting the CoQ10 into a properly chosen digestible carrier fat(s), recrystallization issues are solved and a substrate is supplied for improved action upon CoQ10 by bile acids and lipases. There is no phase separation of the CoQ10 from its carrier fat(s) and the resulting composition easily can be introduced into tablets, capsules and other dry delivery formats.

CoQ10 melts at approximately 49° C. Using the art taught herein, it is possible to melt the CoQ into a fat with a melting point as low as 40° C., as high as 150° C., and into mixtures of digestible fats with a variety of melting points. Below this range, the carrier will tend to be liquid at room temperature and may allow phase separation, encounter tableting difficulties, or be prone to other issues. Above this range, there may be heat degradation of the CoQ10 or unwanted interactions between the CoQ10 and other components of the melt. The art taught here allows for the production of CoQ10 products with superior bioavailability, better stability and shelf life, and utilization via tablets, capsules and other dry delivery modalities, either as a single ingredient or as part of complex formulations. This art also can be used to improve CoQ10 inclusion in a variety of soft gelatin capsule formulations.

### DESCRIPTION OF THE PREFERRED EMBODIMENTS

The present invention pertains to the surprising discovery that ubiquinone (CoQ₁₀) can be melted beneficially into digestible and fully absorbable carrier fats that are solid at room and body temperature. By melting the CoQ₁₀ into a properly chosen digestible carrier fat(s), recrystallization issues are solved and a substrate is supplied for improved action upon CoQ₁₀ by bile acids and lipases. There is no phase separation of the CoQ₁₀ from its carrier fat(s) and the resulting composition easily can be introduced into tablets, capsules and other dry delivery formats.

CoQ₁₀ melts at approximately 49° C. Using the art taught herein, it is possible to melt the CoQ into a fat with a melting point as low as 40° C., as high as 150° C., and into mixtures of digestible fats with a variety of melting points. Below this range, the carrier will tend to be liquid at room temperature and may allow phase separation, encounter tableting difficulties, or be prone to other issues. Above this range, there may be heat degradation of the CoQ₁₀ or unwanted interactions between the CoQ₁₀ and other components of the melt.

The invention makes use of digestible/bioavailable long-chain hydrocarbons as fusible carrier materials. The preferred range for melting is 45° C. to about 90° C. Substances known as waxes, for example, bees wax, are not preferred because these are not digested and therefore cannot serve as true carriers of CoQ₁₀ via micelles. Naturally occurring hydrocarbons that are acceptable for the purposes of the invention include, but are not limited to lauric acid, myristic acid, stearic acid, palmitic acid, palm fruit stearin, solid fractions from the edible oils of plants such as coconut, corn, cottonseed, groundnut, hazelnut, nutmeg, oats, palm and palm kernel, peanut, rice bran, sesame, soy, sunflower and other sources. Hydrogenated oils, such as LubriTab®, suitable fatty acid esters, and similar items may be used by one skilled in the art, as might a mixture of hydrocarbons to achieve desired qualities of melting point, texture, non-phase separation with CoQ₁₀, stability, improved assimilation, workability and stability.

### EXAMPLE 1

A difficult issue with CoQ₁₀ is its tendency to crystallize out of oils, lecithin and other carriers. In the current experiment, 0.5 g Lubritab® hydrogenated vegetable oil (JRS Pharma LP) was used as the fusible carrier. Lubritab® is made from fully hydrogenated refined vegetable oil that is sprayed into a dry, fine powder and is edible. Its melting point is 57-70° C. To start, 0.5 g Lubritab® and 0.5 g ubiquinone (Kanaka) were placed in a sterile 15 ml polystyrene centrifuge tube (Fisher) along with a magnetic stir bar. The tube was heated to 70° C. with constant stirring until both phases were melted and the liquid was homogeneous (approximately 5 minutes). The tube was removed from heat, and allowed to slowly cool to room temperature. The mixture cooled without phase separation into a yellow-orange solid which remained homogeneous by light microscopy after ten days (400× magnification). The solid subsequently was ground into a fine, free-flowing powder.

### EXAMPLE 2

Because of the current interest in trans-fat free materials, naturally saturated fatty acids are of increasing importance. Stearic acid is found in cocoa butter, butter fat, a variety of animal fats, chicken fat, and seed oils. It has a melting point in pure form of 69.6° C. and consists of white crystals or powder. U.S.P stearic acid may be a mixture of stearic and palmitic acids, its crystals or powder may have a slight yellowish cast, and it does not congeal below 54° C. As in Experiment 1, 0.5 g stearic acid and 0.5 g ubiquinone (Kanaka) were placed in a sterile 15 ml polystyrene centrifuge tube (Fisher) along with a magnetic stir bar. The tube was heated to 70° C. with constant stirring until both phases were melted and the liquid was homogeneous (approximately 5 minutes). The tube was removed from heat, and allowed to slowly cool to room temperature. The mixture cooled without phase separation into a yellow-orange solid. The solid subsequently was ground into a fine, free-flowing powder.

### EXAMPLE 3

Palmitic acid is a saturated fatty acid obtainable from both plant an animal sources. Palm oil consists of up to 45% plamitic acid and this makes palm oil an inexpensive source of this material for manufacture. Palmitic acid melts at 63-64° C. and exists in pure form as white crystalline scales. As in Experiment 1, 0.5 g palmitic acid and 0.5 g ubiquinone (Kanaka) were placed in a sterile 15 ml polystyrene centrifuge tube (Fisher) along with a magnetic stir bar. The tube was heated to 65° C. with constant stirring until both phases were melted and the liquid was homogeneous (approximately 5 minutes). The tube was removed from heat, and allowed to slowly cool to room temperature. The mixture cooled without phase separation into a yellow-orange solid. The solid subsequently was ground into a fine, free-flowing powder.

### EXAMPLE 4

The fundamental requirements of the invention are 1) the carrier be digestible and readily assimilate, 2) the carrier prevent recrystallization of the CoQ10, and 3) the carrier not undergo phase separation with the CoQ10. Lauric acid is a medium chain fatty acid without double bonds. It is common in coconut, palm kernel and a number of other oil sources. Lauric acid has a relatively low melting point of 42.2-44° C., which is below that of CoQ10. Using lauric acid as a carrier will depress slightly the melting point of CoQ10 (approximately 49° C.), but the fused materials will remain sold at room temperature. Lauric acid is an acceptable fusible agent, but the resulting product will tend to liquefy during tablet stamping. Therefore, with this and other sold fatty acids with similar melting points, it is useful to lay the resulting material onto micro crystalline cellulose or such materials. Alternatively, a fatty acid with a higher melting point can be fused into the mixture.

### EXAMPLE 5

| Ingredients | Percent | Mg Per Capsule |
|---|---|---|
| CoQ₁₀ | 48.78% | 200.00 |
| LubriTab ® | 48.78% | 200.00 |
| Cellulose | 1.21% | 5.00 |
| Magnesium Stearate | 0.96% | 4.00 |
| Silica | 0.24% | 1.00 |
| Total | 99.97% | 410.00 |

In this simple formula, the first two ingredients are prepared according to the method in Example 1. The last three ingredients are pre-blended and then added to the larger mixture and again blended until uniformly dispersed. The resulting powder is encapsulated at the rate of 410 mg per capsule. With the addition of suitable binding agents, such as dicalcium phosphate, the blend of this example can easily be used in tablet manufacture.

### EXAMPLE 6

| Ingredients | Percent | Mg Per Tablet |
|---|---|---|
| L-Carnitine Fumarate (58% carnitine) | 64.23 | 862.00 |
| CoQ₁₀ | 7.45 | 100.00 |
| LubriTab ® | 7.45 | 100.00 |
| Dicalcium Phosphate | 10.80 | 145.00 |
| Microcrystalline cellulose (MCC) | 7.45 | 100.00 |
| Magnesium Stearate | 0.75 | 10.00 |
| Zein (coating) | 1.86 | 25.00 |
| Total | 99.99 | 1342.00 |

In this formula, the CoQ₁₀ and Lubritab® are prepared according to the method in Example 1 and then mixed with the L-carnitine fumarate. The dicalcium phosphate, MCC and magnesium stearate are mixed separately, then blended into the large batch. The resulting mixture is pressed into tablets and coated with Zein according to standard procedures.

I claim:

1. A composition for facilitating the delivery of coenzyme Q₁₀ wherein said composition comprises:
(i) a first component consisting of coenzyme Q₁₀ in a pharmaceutically acceptable form and
(ii) a second component consisting of a fusible carrier material wherein said composition is comprised of said first component and said second component in a ratio sufficient to prevent phase separation and the recrystallization of the coenzyme Q₁₀.

2. The composition of claim 1 wherein the coenzyme Q₁₀ is selected from the group consisting of coenzyme Q₁₀, reduced coenzyme Q₁₀, semi-reduced coenzyme Q₁₀ and analogs thereof.

3. The composition of claim 1 wherein the fusible carrier material is a digestible and bioavailable fatty acid with a melting point between 40° C. and 150° C.

4. The composition of claim 1 wherein the fusible carrier material is selected from the group consisting of but not limited to: lauric acid, myristic acid, stearic acid, palmitic acid, palm fruit stearin, solid fractions from the edible oils of plants such as coconut, corn, cottonseed, groundnut, hazelnut, nutmeg, oats, palm and palm kernel, peanut, rice bran, sesame, soy, sunflower and other sources; hydrogenated oils, such as LubriTab®; suitable fatty acid esters; suitable mixtures of room temperature solid and liquid fatty acids customized such that the resultant has a melting point between 40° C. and 150° C.; and mixtures of the foregoing.

5. The composition of claim 1 wherein the coenzyme Q₁₀ is fused into said carrier material in an amount of about 0.1% by weight to about 75% by weight.

6. The composition of claim 1 wherein the coenzyme Q₁₀ is fused into said carrier material in an amount of about 5% by weight to about 50% by weight.

7. The composition of claim 1 wherein the composition is used as a component of a capsule, tablet or other dry delivery form.

8. The composition of claim 1 wherein the composition is used as an ingredient delivered via a soft gelatin capsule.
